(19) United States
(12) Patent Application Publication
(10) Pub. No.: US 2022/0199250 A1
(43) Pub. Date: Jun. 23, 2022

(54) APPARATUS FOR REMOTE HEALTHCARE

(71) Applicants: Ankith Neel Chandra, Austin, TX (US); Chandrashekhar Appanna, Austin, TX (US); Srinivasa R. Venkatesh, Houston, TX (US)

(72) Inventors: Ankith Neel Chandra, Austin, TX (US); Chandrashekhar Appanna, Austin, TX (US); Srinivasa R. Venkatesh, Houston, TX (US)

(21) Appl. No.: 17/645,899

(22) Filed: Dec. 23, 2021

Related U.S. Application Data
(60) Provisional application No. 63/130,283, filed on Dec. 23, 2020.

(51) Int. Cl. G16H 40/67; G16H 80/00; G16H 10/60; G06F 3/16

(52) U.S. Cl. CPC G16H 40/67; G16H 80/00; G06F 3/167; H04L 63/08; H04L 63/0428; G16H 10/60

(57) ABSTRACT

In an example, a method may include receiving, by a patient consultation device, a session request from a provider consultation device; determining, by the patient consultation device, whether the received session request is authenticated; in response to a determination that the received session request is authenticated, the patient consultation device establishing a consultation session with the provider consultation device; receiving, by the patient consultation device, an activation request from the provider consultation device; and in response to a receipt of the activation command from the provider consultation device, the patient consultation device activating a stethoscopic microphone.

This application claims priority to U.S. Provisional Patent Application No. 63/130,283, filed on Dec. 23, 2020, in the name of Ankith Neel Chandra, entitled “Method and Apparatus for Audio, Visual and Data Based Remote Healthcare,” the disclosure of which is hereby incorporated by reference.

### TECHNICAL FIELD

Embodiments relate to an electronic device for providing remote healthcare.

### BACKGROUND

Technology based remote healthcare is becoming a necessity. Such remote healthcare may be implemented for situations in which direct in-person care may be difficult or impractical. For example, such situations may include remote rural locations, lack of transport, lack of mobility, quarantine conditions, lack of funding for medical facilities and personnel, pandemics, and so forth.

### BRIEF DESCRIPTION OF THE DRAWINGS

Some implementations are described with respect to the following figures.

FIG. 1 is a schematic diagram of an example system, in accordance with some implementations.

FIG. 2 is a schematic diagram of an example consultation device, in accordance with some implementations.

FIGS. 3A-3B are schematic diagrams of example consultation devices, in accordance with some implementations.

FIG. 4 is a flow diagram of an example process, in accordance with some implementations.

FIG. 5 is a schematic diagram of an example computing device, in accordance with some implementations.

FIG. 6 is a flow diagram of an example process, in accordance with some implementations.

FIG. 7 is a diagram of an example machine-readable medium storing instructions in accordance with some implementations.

Throughout the drawings, identical reference numbers designate similar, but not necessarily identical, elements. The figures are not necessarily to scale, and the size of some parts may be exaggerated to more clearly illustrate the example shown. Moreover, the drawings provide examples and/or implementations consistent with the description; however, the description is not limited to the examples and/or implementations provided in the drawings.

### DETAILED DESCRIPTION

In the present disclosure, use of the term “a,” “an,” or “the” is intended to include the plural forms as well, unless the context clearly indicates otherwise. Also, the term “includes,” “including,” “comprises,” “comprising,” “have,” or “having” when used in this disclosure specifies the presence of the stated elements, but do not preclude the presence or addition of other elements.

As people age, the need for regular and frequent medical examinations becomes increasingly important. However, for many senior citizens, the process of consultation with a medical provider may be cumbersome and inconvenient. For example, some senior citizens may find it difficult to use email and web interfaces to schedule medical appointments. Further, some senior citizens may find it difficult to keep track of multiple appointments with different providers, of referrals to specialists, and so forth. In addition, some senior citizens may have difficulty in driving a car, navigating to different medical offices, parking, and so forth. These various difficulties may be reduced conducting consultations via remote healthcare technology that is transparent and easy to use.

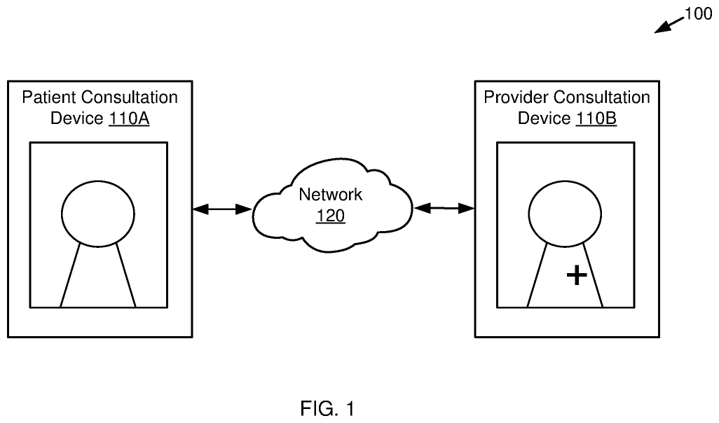

FIG. 1—Example System

Referring now to FIG. 1, shown is an example remote healthcare system 100, in accordance with some implementations. As shown, the example remote healthcare system 100 may include a patient consultation device 110A coupled to a provider consultation device 110B via a network 120. Each of the consultation devices 110A, 110B (also referred to herein as “consultation devices 110”) may be electronic device to provide a remote healthcare consultation between a patient and a medical provider. In some embodiments, the consultation devices 110 may provide two-way audio and video communications between the patient and the provider. Further, the consultation devices 110 may include functionality to allow the provider to perform remote medical observations of the patient. Such medical observations may include measurement of blood pressure, measurement of heart rate, listening to internal body sounds (i.e., auscultation), and so forth. In some embodiments, the patient consultation device 110A and the provider consultation device 110B may be substantially identical devices (i.e., including the same hardware components).

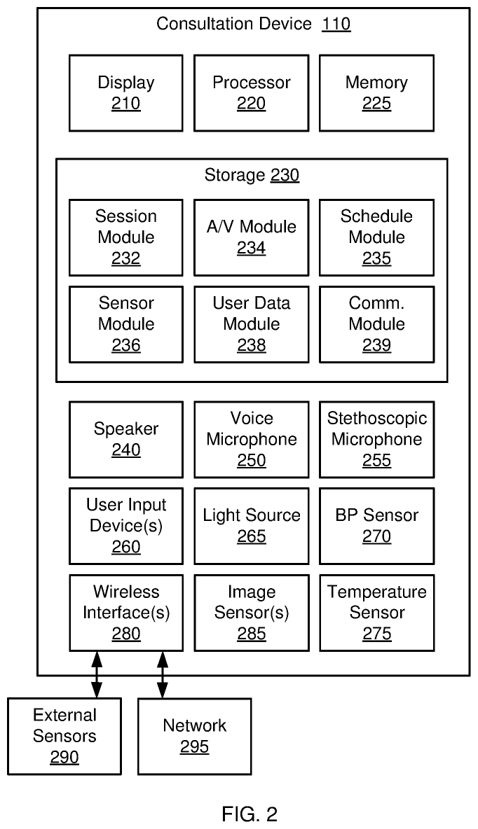

FIG. 2—Example Consultation Device

Referring now to FIG. 2, shown is an example consultation device 110, in accordance with some implementations. As shown, the example consultation device 110 may include a display 210, a processor 220, memory 225, storage 230, a speaker 240, a voice microphone 250, a stethoscopic microphone 255, user input device(s) 160, a light source 265, a blood pressure (BP) sensor 270, a wireless interface(s) 280, image sensor(s) 285, and a temperature sensor 275.

In some embodiments, the processor 220 may be a hardware processing device (e.g., a central processing unit (CPU), a System on a Chip (SoC), a controller, and so forth). The memory 225 may include any type(s) of computer memory (e.g., dynamic random-access memory (DRAM), static random-access memory (SRAM), non-volatile memory (NVM), a combination of DRAM and NVM, etc.). The storage 230 may be a non-transitory machine-readable medium to store computer data and instructions, such as an optical, semiconductor, or magnetic storage device.

In one or more embodiments, the display 210 may be a display device such as a liquid crystal display (LCD), a light-emitting diode (LED) display, a projector, and so forth. Further, the image sensor(s) 285 may include a camera charge-coupled device (CCD), a complementary metal-oxide-semiconductor (CMOS) sensor, and so forth. The speaker 240 may provide audio output from a received signal. Further, the voice microphone 250 may be a sound sensor configured to convert audible sounds such as words spoken by a user of the consultation device 110. In some embodiments, the display 210, image sensor(s) 285, the speaker 240, and the voice microphone 250 may be used to provide two-way video and audio communication with another consultation device 110 (e.g., between the patient and provider consultation devices 110A, 110B shown in FIG. 1).

In some embodiments, the user input device(s) 160 may include one or more sensors to receive user inputs to control the consultation device 110 (e.g., buttons, touch sensitive surfaces, and so forth). The light source 265 may include an LED light, an incandescent light, and so forth. The temperature sensor 275 may be a sensor configured to obtain temperature readings from portions of a human body (e.g., infrared thermometer, thermocouple, etc.).

In some embodiments, the wireless interface(s) 280 may provide on or more wireless connections to remote sensors 290 and/or a network 295. For example, the wireless interface(s) 280 may include a Bluetooth interface, a cellular telephony interface, a WiFi interface, a near-field communication (NFC) interface, and so forth. In some examples, the wireless interface(s) 280 may allow a user to connect to the consultation device using a wireless headset or earphone. In some embodiments, the consultation device 110 may be implemented on a smartphone hardware platform.

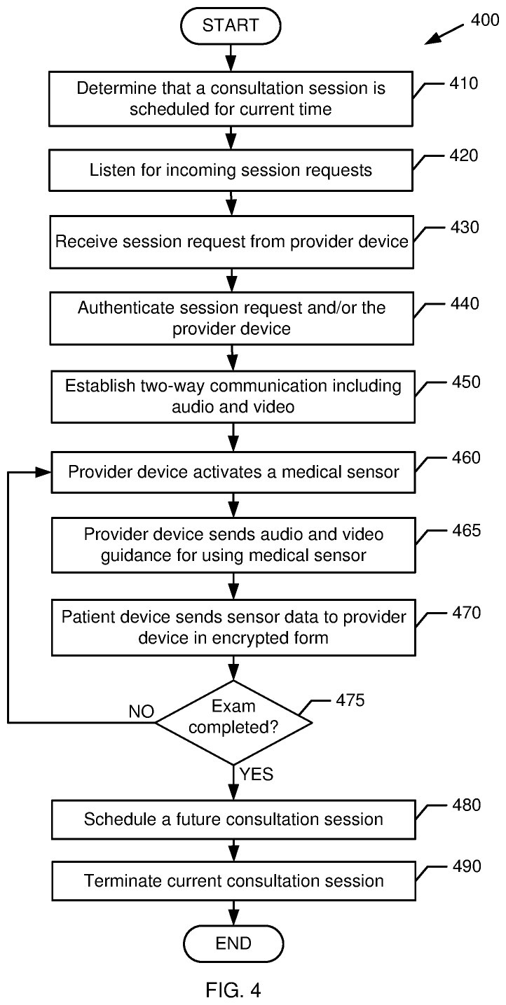

In one or more embodiments, the stethoscopic microphone 255 may be a specialized sensor to perform auscultation (i.e., listen to internal body sounds). For example, the stethoscopic microphone 255 may be configured to be placed in direct contact with a human body, and thereby detect heart sounds, lung sounds, and so forth. An example implementation of the stethoscopic microphone 255 is described below with reference to FIG. 4.

In some embodiments, the blood pressure (BP) sensor 270 may include one or more sensing devices to measure the blood pressure in a human body. For example, the BP sensor 270 may be implemented as a transdermal optical image sensor that uses LED light and software processing to obtain a BP measurement. In another example, the BP sensor 270 may include a device that applies physical pressure to a portion of the human body (e.g., a wrist cuff, a finger cuff, etc.). In still another example, the BP sensor 270 may use an image sensor and light source (e.g., image sensor(s) 285 and light source 265) and software to obtain a BP measurement adjusted for the user's age, height, weight, and so forth. In some embodiments, the BP sensor 270 may be included in (or disposed on) the consultation device 110. However, in other embodiments, the BP sensor 270 may be implemented as a remote sensor 290 connected to the wireless interface(s) 280. For example, the BP sensor 270 may be a battery-powered wrist cuff that is paired to establish a wireless connection (e.g., a Bluetooth connection) to the consultation device 110.

In one or more embodiments, the storage 230 may include various software modules, including a session module 232, an audio/visual (A/V) module 234, a schedule module 235, a sensor module 236, a user data module 238, and a communications module 239. These modules may include stored instructions that are executable by the processor 220. In some embodiments, the communications module 239 may control communication of the consultation device 110. For example, the communications module 239 may control the wireless interface(s) 280 to connect to a cellular network, a WiFi network, a Bluetooth device, a NFC device, and so forth.

In some embodiments, the session module 232 may be executed to establish a secure consultation session between patient and provider devices (e.g., between the patient and provider consultation devices 110A, 110B shown in FIG. 1). For example, the session module 232 may determine that a consultation session is scheduled to start at the current time, or in a given time period (e.g., in the next five minutes, the next ten minutes, etc.). In response to this determination, the session module 232 may begin to actively listen or monitor for incoming session requests from a remote device ((e.g., from provider consultation device 110B shown in FIG. 1). Upon receiving a session request, the session module 232 may authenticate the session request and/or the remote device (e.g., by validating a password, a cryptographic signature, a certificate, and so forth), and may then establish the consultation session with the remote device. Once the session is established, the A/V module 234 may be executed to provide two-way audio and video communication between the consultation device 110 and the remote device (e.g., to allow the patient and the provider to see each other and speak to each other).

Note that, while the session module 232 may listen for incoming session requests, embodiments are not limited in this regard. For example, it is contemplated that the session module 232 may allow a patient device to send a session request to a provider device (e.g., in response to a user command, at the time of a scheduled session, etc.). Further, in some examples, both the patient device and the provider may begin listening for session requests, and the session requests may be sent by either device. Other variations are possible.

In one or more embodiments, the schedule module 235 may be executed to allow a user to schedule and coordinate consultation sessions with different providers (e.g., providers that support the consultation device 110, providers that are authorized to interact with the consultation device 110, and so forth). For example, the schedule module 235 may allow a user to view a calendar of scheduled sessions, a list of available providers, a calendar of available time slots for scheduling additional sessions, and so forth. Further, the schedule module 235 may provide reminders of upcoming scheduled sessions to the user of the consultation device 110. In some examples, the schedule module 235 may notify the session module 232 of a session that is due to start (e.g., to cause the session module 232 to begin listening for incoming calls). In some embodiments, the schedule module 235 may track the medical history and preferences of the patient for use in scheduling events. For example, the schedule module 235 may recommend times for sessions based on the patient's usual wake-up time, usual medicine intake time, typical caregiver arrival time, and so forth. Further, the schedule module 235 may provide reminders that a medicine intake time is due, that a caregiver arrival time is due, etc.

In some embodiments, the sensor module 236 may be executed to control various sensors of the consultation device 110, such as the stethoscopic microphone 255, the BP sensor 270, the temperature sensor 275, external sensors 290, and so forth. Further, in some embodiments, the sensor module 236 may activate a particular sensor under specific conditions. For example, the sensor module 236 may only activate the stethoscopic microphone 255 or the BP sensor 270 of a patient consultation device 110 in response to an activation command received from a remote provider device 110 (e.g., a command entered by a provider during a consultation session). In other examples, the sensor module 236 may activate sensors based on predetermined programming, an expert system, a patient command, and so forth. In one or more embodiments, the sensor module 236 may collect data provided by the sensors, and may cause the sensor data to be stored in the storage 230.

In one or more embodiments, the user data module 238 may be executed to store and protect sensitive and medical data in the consultation device 110. For example, the user data module 238 may encrypt sensor data collected by the sensor module 236, user identification information, financial information, diagnosis information, treatment information, and so forth. Further, in some examples, the user data module 238 may store an encrypted log of activity by the user of the consultation device 110 (e.g., movement information, number of sessions conducted, treatment recommendations and actions, etc.). In some embodiments, the user data module 238 may securely transfer some or all of the user data to a provider consultation device 110B. For example, upon establishing a consultation session with a particular provider, the user data module 238 may identify stored sensor data that is relevant to the provider, authenticate the provider consultation device 110B, and then transmit the identified data to the provider consultation device 110B in encrypted form. The user data module 238 may encrypt and decrypt stored and transmitted user data using one or more cryptographic or encryption techniques (e.g., TLS, IPSec, etc.). In some examples, the user data module 238 may request explicit approval from the user (i.e., patient) before transmitting any of the user's data to a remote device.

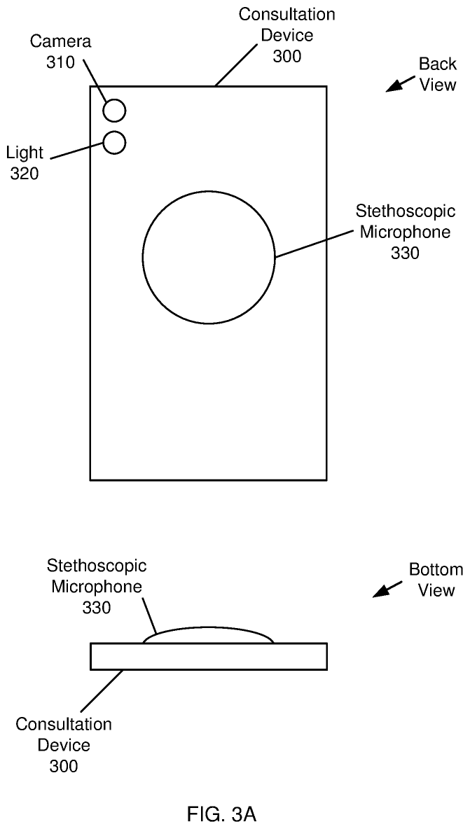

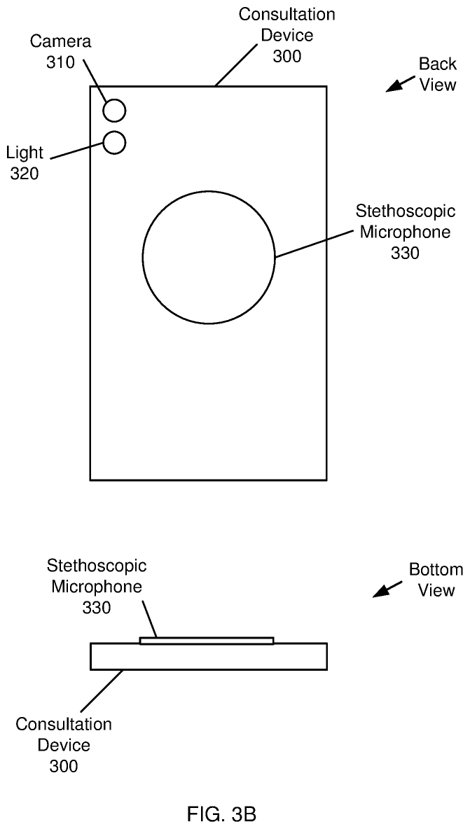

FIGS. 3A-3B—Example Consultation Devices

Referring now to FIG. 3A, shown is a first example consultation device 300, in accordance with some implementations. As shown, the consultation device 300 may include a camera 310, a light 320, and a stethoscopic microphone 330. The consultation device 300, camera 310, light 320, and stethoscopic microphone 330 may respectively correspond to example implementations of the consultation device 110, image sensor(s) 285, light source 265, and stethoscopic microphone 255 shown in FIG. 2.

In some embodiments, the stethoscopic microphone 330 may be located on a back outer face of the consultation device 300. Further, the stethoscopic microphone 330 may have an outer surface or cover that is substantially convex and circular (e.g., a dome-like surface). As used herein, the “back” outer face of the stethoscopic microphone 330 may refer to the surface that is opposite to a “front” face that includes the main display of the stethoscopic microphone 330 (not shown in FIG. 3). As shown, in some embodiments, the curved surface of the stethoscopic microphone 330 may project outward from the consultation device 300, thereby allowing close contact of the stethoscopic microphone 330 with a particular location on the human body.

Referring now to FIG. 3B, shown is a second example consultation device 300, in accordance with some implementations. As shown in the FIG. 3B, in some embodiments, the stethoscopic microphone 330 may have a flat surface that is substantially parallel to the back outer face of the consultation device 300. Other variations are possible.

In one or more embodiments, the stethoscopic microphone 330 shown in FIGS. 3A-3B may be activated by a command received from a provider consultation device (e.g., provider consultation device 110B shown in FIG. 1). Further, in some embodiments, the consultation device 300 may display (e.g., in display 210) a still or video image to instruct the patient of the proper location(s) on the patient's body which should be in contact with the stethoscopic microphone 330. The stethoscopic microphone 330 may then detect auscultation sounds from the patient's body at the desired location. The consultation device 300 may transmit an electronic signal representing the auscultation sounds to the provider device, and may thereby allow the provider to listen to the auscultation sounds in near real-time fashion. In some embodiments, the consultation device 300 may output audio and/or video instructions from the provider to ask the patient to move the placement of the stethoscopic microphone 330 as needed.

In some embodiments, the provider may use the camera 310 to determine the current location of the stethoscopic microphone 330. The provider may then provide instructions to the patient regarding changes to the placement of the stethoscopic microphone 330.

FIG. 4—Example Process

Referring now to FIG. 4, shown is an example process 400, in accordance with some implementations. The process 400 may be performed by the processor 220 executing instructions (e.g., one or more of the modules included in storage 230), as shown in FIG. 2. The process 400 may be implemented in hardware or a combination of hardware and programming (e.g., machine-readable instructions executable by a processor(s)). The machine-readable instructions may be stored in a non-transitory computer readable medium, such as an optical, semiconductor, or magnetic storage device. The machine-readable instructions may be executed by a single processor, multiple processors, a single processing engine, multiple processing engines, and so forth. For the sake of illustration, details of the process 400 may be described below with reference to FIGS. 1-2, which show examples in accordance with some implementations. However, other implementations are also possible.

Block 410 may include determining that a consultation session is scheduled for current time. Block 420 may include listening for incoming session requests. For example, referring to FIGS. 1-2, the session module 232 of the patient consultation device 110A may determine that a consultation session is scheduled to start at the current time, and may then begin to actively monitor for incoming session requests from the provider consultation device 110B.

Referring again to FIG. 4, block 430 may include receiving a session request from a provider device. Block 440 may include authenticating the session request and/or the provider device. Block 450 may include establishing two-way communication including audio and video. For example, referring to FIGS. 1-2, the session module 232 of the patient consultation device 110A may receive a session request from the provider consultation device 110B. The session module 232 may authenticate the session request, and then establish a consultation session with the provider consultation device 110B. Once the consultation session is established, the A/V module 234 may provide two-way audio and video communication between the patient consultation device 110A and the provider consultation device 110B. For example, a doctor may use the display 210 of the provider consultation device 110B to perform a visual examination of the patient (e.g., as captured by an image sensor 285 of the patient consultation device 110A). In another example, the doctor and the patient may be able to speak and listen to each other via the speaker 240 and voice microphone 250 of their respective consultation devices 110.

Referring again to FIG. 4, block 460 may include the provider device activating a medical sensor. Block 465 may include the provider device sending audio and/or video guidance for using the medical sensor. For example, referring to FIGS. 1-3, a doctor or other provider may interact with the provider consultation device 110B to transmit a sensor activation signal to the patient consultation device 110A. The sensor module 236 of the patient consultation device 110A may receive the sensor activation signal, and in response may activate the stethoscopic microphone 300. Further, in some embodiments, the provider may interact with the provider consultation device 110B to transmit instruction information (e.g., image, video, text, audio, animation, etc.) to inform the patient on the proper use of the stethoscopic microphone 300. The patient consultation device 110A may then present the instruction information to the patient. For example, the screen 210 of the patient consultation device 110A may display an illustration indicating the proper location(s) on the patient's body at which the stethoscopic microphone 300 should be pressed or otherwise located.

Referring again to FIG. 4, block 470 may include the patient device sending sensor data to provider device in encrypted form. Decision block 475 may include determining whether a medical examination of the patient is complete. If not, the process 400 may return to block 460 (i.e., to activate another medical sensor for use in the examination). For example, referring to FIGS. 1-3, the user data module 238 of the patient consultation device 110A may encrypt data collected by the sensor module 236 (e.g., sensor data from BP sensor 270, temperature sensor 275, external sensors 290, stethoscopic microphone 255, etc.). Further, in some examples, the user data module 238 may transmit the encrypted sensor data to the provider consultation device 110B. In some examples, the user data module 238 may request explicit approval from the patient (e.g., via a graphical user interface) before transmitting any of the user's data to a remote device. The provider may interact with the provider consultation device 110B to transmit additional sensor activation signal(s) to the patient consultation device 110A. In this manner, the provider may activate and use multiple medical sensors to perform various observations and/or tests as needed during the consultation session.

Referring again to FIG. 4, if it is determined at decision block 475 that the medical examination of the patient is complete, the process 400 may continue at block 480, including scheduling a future consultation session. Block 490 may include terminating the current consultation session. After block 490, the process 400 may be completed. For example, referring to FIGS. 1-2, the patient and/or the provider may interact with the schedule module 235 of their respective consultation devices 110 to schedule the next consultation session. In some examples, the schedule module 235 may display a calendar including a plurality of available time slots, receive a user selection of a particular time slot of the available time slots, and schedule a future consultation session in the particular time slot. The schedule module 235 may provide a reminder of the future consultation session when the scheduled time slot is approaching. Further, in some examples, the schedule module 235 may also provide a calendar of scheduled sessions, a list of available providers, and so forth.

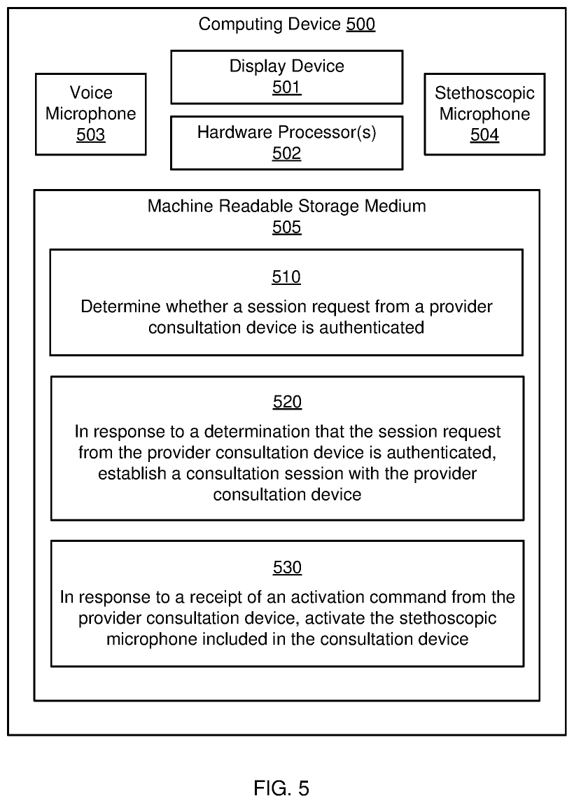

FIG. 5—Example Computing Device

FIG. 5 shows a schematic diagram of an example computing device 500. In some examples, the computing device 500 may correspond to an example implementation of the consultation device 110 (shown in FIG. 1). As shown, the computing device 500 may include a display device 501, a hardware processor 502, a voice microphone 503, a stethoscopic microphone 504, and machine-readable storage 505 including instructions 510-530. The machine-readable storage 505 may be a non-transitory medium. The instructions 510-530 may be executed by the hardware processor 502, or by a processing engine included in hardware processor 502.

Instruction 510 may be executed to determine whether a session request from a provider consultation device is authenticated. Instruction 520 may be executed to, in response to a determination that the session request from the provider consultation device is authenticated, establish a consultation session with the provider consultation device. Instruction 530 may be executed to, in response to a receipt of an activation command from the provider consultation device, activate the stethoscopic microphone included in the consultation device.

For example, referring to FIGS. 1-2, the session module 232 of the patient consultation device 110A may authenticate a session request received from the provider consultation device 110B, and then establish a consultation session with the provider consultation device 110B. Further, the provider may interact with the provider consultation device 110B to transmit a sensor activation signal to the patient consultation device 110A. The sensor module 236 of the patient consultation device 110A may receive the sensor activation signal, and in response may activate the stethoscopic microphone 300.

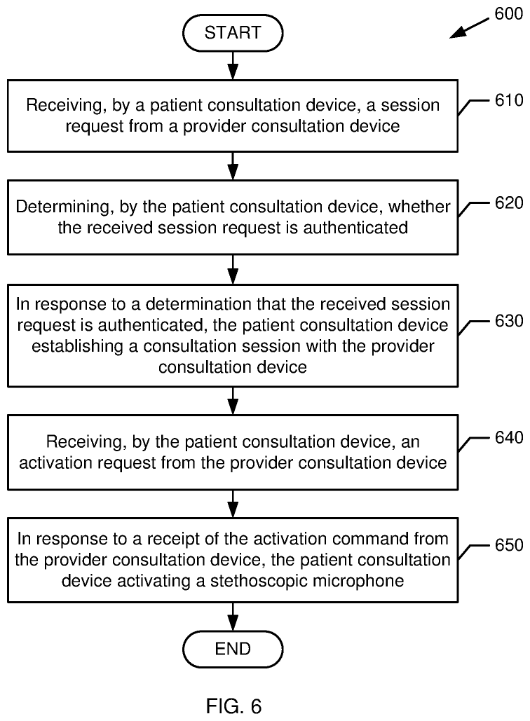

FIG. 6—Example Process

Referring now to FIG. 6, shown is an example process 600, in accordance with some implementations. The process 600 may be performed by the processor 220 executing instructions (e.g., one or more of the modules included in storage 230), as shown in FIG. 2. The process 600 may be implemented in hardware or a combination of hardware and programming (e.g., machine-readable instructions executable by a processor(s)). The machine-readable instructions may be stored in a non-transitory computer readable medium, such as an optical, semiconductor, or magnetic storage device. The machine-readable instructions may be executed by a single processor, multiple processors, a single processing engine, multiple processing engines, and so forth. For the sake of illustration, details of the process 600 may be described below with reference to FIGS. 1-2, which show examples in accordance with some implementations. However, other implementations are also possible.

Block 610 may include receiving, by a patient consultation device, a session request from a provider consultation device. Block 620 may include determining, by the patient consultation device, whether the received session request is authenticated. Block 630 may include, in response to a determination that the received session request is authenticated, the patient consultation device establishing a consultation session with the provider consultation device. Block 640 may include receiving, by the patient consultation device, an activation request from the provider consultation device. Block 650 may include, in response to a receipt of the activation command from the provider consultation device, the patient consultation device activating a stethoscopic microphone.

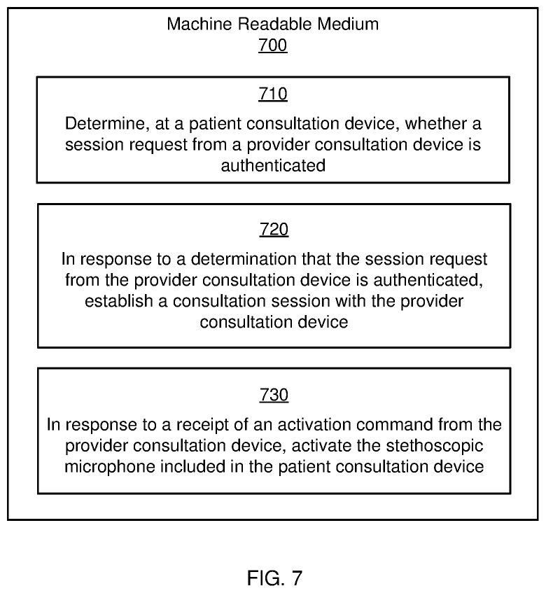

FIG. 7—Example Machine-Readable Medium

FIG. 7 shows a machine-readable medium 700 storing instructions 710-730, in accordance with some implementations. The instructions 710-730 can be executed by a single processor, multiple processors, a single processing engine, multiple processing engines, and so forth. The machine-readable medium 700 may be a non-transitory storage medium, such as an optical, semiconductor, or magnetic storage medium.

Instruction 710 may be executed to determine, at a patient consultation device, whether a session request from a provider consultation device is authenticated. Instruction 720 may be executed to, in response to a determination that the session request from the provider consultation device is authenticated, establish a consultation session with the provider consultation device. Instruction 730 may be executed to, in response to a receipt of an activation command from the provider consultation device, activate the stethoscopic microphone included in the patient consultation device.

In accordance with implementations described herein, a remote healthcare system may include electronic devices to provide consultations between patient and medical providers. In some embodiments, the consultation devices may provide two-way audio and video communications between the patient and the provider. Further, the consultation devices may include functionality to allow the provider to perform remote medical observations of the patient. Such medical observations may include measurement of blood pressure, measurement of heart rate, listening to internal body sounds (i.e., auscultation), and so forth. In this manner, one or more embodiments may provide medical consultations using technology that is transparent and easy to use.

Note that, while FIGS. 1-7 show various examples, implementations are not limited in this regard. For example, referring to FIG. 2, it is contemplated that the consultation device 110 may include additional devices and/or components, fewer components, different components, different arrangements, and so forth. In another example, it is contemplated that the functionality of the consultation device 110 described above may be included in any another engine or software of the consultation device 110. Other combinations and/or variations are also possible.

Data and instructions are stored in respective storage devices, which are implemented as one or multiple computer-readable or machine-readable storage media. The storage media include different forms of non-transitory memory including semiconductor memory devices such as dynamic or static random access memories (DRAMs or SRAMs), erasable and programmable read-only memories (EPROMs), electrically erasable and programmable read-only memories (EEPROMs) and flash memories; magnetic disks such as fixed, floppy and removable disks; other magnetic media including tape; optical media such as compact disks (CDs) or digital video disks (DVDs); or other types of storage devices.

Note that the instructions discussed above can be provided on one computer-readable or machine-readable storage medium, or alternatively, can be provided on multiple computer-readable or machine-readable storage media distributed in a large system having possibly plural nodes. Such computer-readable or machine-readable storage medium or media is (are) considered to be part of an article (or article of manufacture). An article or article of manufacture can refer to any manufactured single component or multiple components. The storage medium or media can be located either in the machine running the machine-readable instructions, or located at a remote site from which machine-readable instructions can be downloaded over a network for execution.

In the foregoing description, numerous details are set forth to provide an understanding of the subject disclosed herein. However, implementations may be practiced without some of these details. Other implementations may include modifications and variations from the details discussed above. It is intended that the appended claims cover such modifications and variations.

What is claimed is:

1. A consultation device comprising:
a display device;
a voice microphone to receive a voice input;
a stethoscopic microphone to receive auscultatory sounds via direct contact with a human body; and
a processor to:
determine whether a session request from a provider consultation device is authenticated;
in response to a determination that the session request from the provider consultation device is authenticated, establish a consultation session with the provider consultation device;
in response to a receipt of an activation command from the provider consultation device, activate the stethoscopic microphone included in the consultation device.

2. The consultation device of claim 1, wherein the display device is to provide an image to indicate proper placement of the stethoscopic microphone on one or more locations on a body of a patient.

3. The consultation device of claim 2, further comprising an image sensor to capture an image, wherein the provider consultation device is to receive the image and display the image to a provider, and wherein the provider is to determine a current location of the stethoscopic microphone based on the image.

4. The consultation device of claim 3, further comprising a speaker device, wherein the speaker device is to provide audio instructions from the provider to guide the proper placement of the stethoscopic microphone on the one or more locations on the body of the patient.

5. The consultation device of claim 1, further comprising:
a blood pressure sensor to measure blood pressure in the body of the patient.

6. The consultation device of claim 5, wherein the processor is further to:
in response to a receipt of a second activation command from the provider consultation device, activate the blood pressure sensor included in the consultation device.

7. The consultation device of claim 1, wherein the processor is further to:
receive sensor data from at least one medical sensor included in the consultation device;
encrypt the sensor data; and
transmit the encrypted sensor data to the provider consultation device.

8. The consultation device of claim 1, wherein the processor is further to:
track a medical history and preferences of a patient; and
recommend time slots for future sessions based at least on the medical history and preferences of the patient.

9. The consultation device of claim 1, further comprising:
a wireless interface to connect to at least one remote sensor.

10. A method comprising:
receiving, by a patient consultation device, a session request from a provider consultation device;
determining, by the patient consultation device, whether the received session request is authenticated;
in response to a determination that the received session request is authenticated, the patient consultation device establishing a consultation session with the provider consultation device;
receiving, by the patient consultation device, an activation request from the provider consultation device; and
in response to a receipt of the activation command from the provider consultation device, the patient consultation device activating a stethoscopic microphone.

11. The method of claim 10, further comprising:
providing, on a display device of the patient consultation device, a first image indicating proper placement of the stethoscopic microphone on one or more locations on a body of a patient.

12. The method of claim 11, further comprising:
capturing, by an image sensor of the patient consultation device, a second image of the body of the patient;
transmit the second image to the provider consultation device; and
display the second image on a display of the provider consultation device, wherein a provider is to determine a current location of the stethoscopic microphone based on the image.

13. The method of claim 11, further comprising:
providing, by a speaker of the patient consultation device, audio instructions from the provider to guide the proper placement of the stethoscopic microphone on the one or more locations on the body of the patient.

14. The method of claim 10, further comprising:
receiving, by the patient consultation device, a second activation request from the provider consultation device; and
in response to a receipt of the second activation command from the provider consultation device, the patient consultation device activating a blood pressure sensor included in the patient consultation device.

15. A non-transitory machine-readable medium storing instructions that upon execution cause a processor to:
determine, at a patient consultation device, whether a session request from a provider consultation device is authenticated;
in response to a determination that the session request from the provider consultation device is authenticated, establish a consultation session with the provider consultation device; and
in response to a receipt of an activation command from the provider consultation device, activate the stethoscopic microphone included in the patient consultation device.

16. The non-transitory machine-readable medium of claim 15, including instructions that upon execution cause the processor to:
display, on a display device of the patient consultation device, a first image indicating proper placement of the stethoscopic microphone on one or more locations on a body of a patient.

17. The non-transitory machine-readable medium of claim 16, including instructions that upon execution cause the processor to:
capture, using an image sensor of the patient consultation device, a second image of the body of the patient; and
transmit the second image to the provider consultation device, wherein the second image is to be displayed on the provider consultation device, and wherein a provider is to determine a current location of the stethoscopic microphone based on the image.

18. The non-transitory machine-readable medium of claim 16, including instructions that upon execution cause the processor to:
provide, using a speaker of the patient consultation device, audio instructions from the provider to guide the proper placement of the stethoscopic microphone on the one or more locations on the body of the patient.

19. The non-transitory machine-readable medium of claim 15, including instructions that upon execution cause the processor to:
receive, by the patient consultation device, a second activation request from the provider consultation device; and
in response to a receipt of the second activation command from the provider consultation device, activate a blood pressure sensor included in the patient consultation device.

20. The non-transitory machine-readable medium of claim 15, including instructions that upon execution cause the processor to:
receive sensor data from at least one medical sensor included in the patient consultation device;
encrypt the sensor data; and
transmit the encrypted sensor data to the provider consultation device.
